(19) United States
(12) Patent Application Publication
(10) Pub. No.: US 2017/0224976 A1
(43) Pub. Date: Aug. 10, 2017

(54) MANIFOLD CONNECTION ASSEMBLY HAVING A SURFACE FINISH

(71) Applicant: ACIST MEDICAL SYSTEMS, INC., Eden Prairie, MN (US)

(72) Inventor: Matt Bakken, Bloomington, MN (US)

(21) Appl. No.: 15/019,663

(22) Filed: Feb. 9, 2016

(51) Int. Cl. A61M 39/10; A61M 5/145; A61M 39/24; A61M 5/00; F16L 41/02; F16L 55/07

(52) U.S. Cl. CPC A61M 39/1011; F16L 41/02; F16L 55/07; A61M 39/24; A61M 5/007; A61M 5/14546; A61M 2039/1033

(57) ABSTRACT

A manifold associated with a contrast injector system. The manifold may include a first fluid inlet, a second fluid inlet, a fluid outlet, a main passageway, and a valve. The first fluid inlet, second fluid inlet, and fluid outlet may be in fluid communication with the main passageway. The first fluid inlet, second fluid inlet, or fluid outlet can be connected to a conduit for communicating fluid, such as a fluid at high pressure. At least one of the first fluid inlet, second fluid inlet, or fluid outlet can have a surface finish that is in contact with the conduit.

### TECHNICAL FIELD

This disclosure relates to manifold valve assemblies for powered injectors.

### BACKGROUND

Many medical procedures, such as angiographies, involve injecting a contrast media directly into a patient. Angiography is a procedure used in the diagnosis and treatment of cardiovascular conditions including abnormalities or restrictions in blood vessels. During angiography, a radiographic image of the heart or vascular structure is obtained by injecting contrast media through a catheter into a vein or artery of the patient. The injected contrast media can pass to vascular structures in fluid communication with the vein or artery in which the injection is made. X-rays are passed through the region of the body in which the contrast media was injected. The X-rays are absorbed by the contrast media, causing a radiographic outline or image of the blood vessel containing the contrast media.

### SUMMARY

In general, this disclosure is directed to embodiments of a manifold assembly, including a manifold assembly having two fluid inlets and one fluid outlet in fluid communication with a main passageway. Saline may be delivered through the first fluid inlet, and contrast media may be delivered through the second fluid inlet at a high pressure. In some embodiments, the manifold assembly may include a valve configured to switch between allowing fluid communication through either the first fluid inlet or the second fluid inlet, and the fluid outlet. Each inlet and outlet can be connected to a conduit for fluid communication. Depending on the fluid communicated, this connection may need to withstand high pressures. In some embodiments, at least one of the fluid inlets or outlet may have surface finish in contact with the conduit. This surface finish can help make a better connection between the conduit and fluid inlet or outlet.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a perspective view illustrating a contrast injector system having a manifold assembly in accordance with an embodiment of the invention.

FIG. 2A is a diagram illustrating a first operation of a contrast injector system.

FIG. 2B is a diagram illustrating a second operation of a contrast injectors system.

FIG. 2C is a diagram illustrating a third operation of a contrast injector system.

FIG. 3A is a side sectional view illustrating the operation of an inlet valve system and manifold during a first operation.

FIG. 3B is a side sectional view illustrating the operation of an inlet valve system and manifold during a second operation.

FIG. 3C is a side sectional view illustrating the operation of an inlet valve system and manifold during a third operation.

FIG. 3D is a side sectional view illustrating the operation of an inlet valve system and manifold during a fourth operation.

FIG. 4A is a side section view illustrating the operation of an inlet valve system during a first operation of a contrast injector system.

FIG. 4B is a side section view illustrating the operation of an inlet valve system during a second operation of a contrast injector system.

FIG. 4C is a side section view illustrating the operation of an inlet valve system during a third operation of a contrast injector system.

FIG. 5A is a perspective view illustrating a manifold in accordance with an embodiment of the invention.

FIG. 5B is a cross-sectional view illustrating a connection between a conduit and a fluid inlet of a manifold in accordance with an embodiment of the invention.

FIG. 5C is a perspective view illustrating a fluid inlet of a manifold in accordance with an embodiment of the invention.

FIG. 5D is a perspective view illustrating a surface finish on a fluid inlet of a manifold in accordance with an embodiment of the invention.

### DETAILED DESCRIPTION

Various exemplary embodiments are described herein with reference to the accompanying drawing figures in which like numbers describe like elements.

Embodiments of the invention include a manifold having a surface finish adapted to reduce leakage between a connection and reduce forces required to disassemble the connection, particularly connections subjected to high pressures during use. Embodiments also include injector systems having such a manifold, as described further below. Such a manifold can be used with any contrast injector system, including the CVi contrast injector system offered by ACIST Medical Systems, Inc., of Eden Prairie, Minn. Embodiments of a contrast injector system will be described, followed by a description of a manifold having a surface finish. The pertinent parts of U.S. Pat. No. 6,656,157, titled Infinitely Refillable Syringe, which describes contrast injector systems, are hereby incorporated by reference.

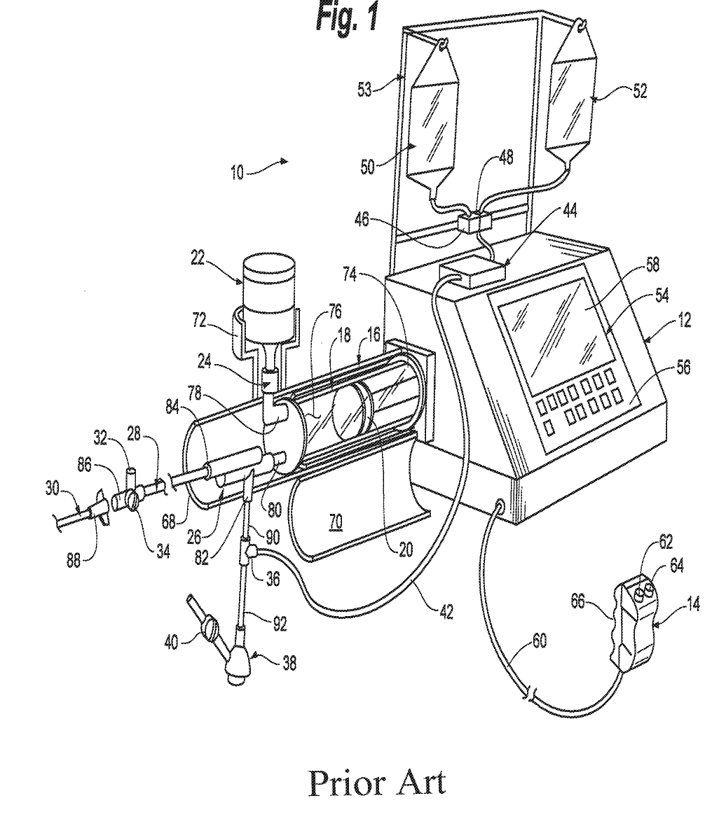

FIG. 1 shows a contrast media injector system 10 for injecting contrast media into a blood vessel under interactive physician control during a medical procedure, such as an angiogram. As shown, system 10 includes main console 12, hand held remote control 14, syringe holder 16, syringe main body 18, syringe plunger/piston 20, radiographic material reservoir (bottle) 22, inlet valve system 24, manifold 26, high pressure tube 28, catheter 30, patient medication port 32, three-way stop-cock 34, T-connector 36, pressure transducer 38, stop-cock 40, tubing 42, peristaltic pump 44, saline check valve 46, waste check valve 48, saline bag 50, waste bag 52, and bag support rack 53. It should be noted that system 10 is just one embodiment of contrast media injector system in accordance with the invention. Other embodiments may include a second syringe holder, syringe main body, and syringe plunger in communication with a saline bag instead of the peristaltic pump shown in FIG. 1.

In the embodiments shown, console 12 houses the electrical controls for system 10, together with the motors which drive piston/plunger 20 and peristaltic pump 44. On the front surface of console 12, user interface 54 provides control switches 56 and display 58 through which the user may enter control settings and monitor the operational state of system 10.

Remote control 14 can be connected to console 12 by cable 60 (although in other embodiments remote control 14 may be connected by a wireless connection such as an RF, infrared optic, or ultrasonic link). Remote control 14 is, in the embodiment shown in FIG. 1, a hand-held control which includes reset and saline push button switches 62 and 64, respectively, and flow rate control lever or trigger 66. By squeezing trigger 66, the user can provide a command signal to console 12 to provide a continuously variable injection rate.

As shown in FIG. 1, syringe holder 16 projects from the left hand side of console 12. Syringe holder 16 is preferably a clear material, and includes a half cylindrical back shell 68, a half cylindrical front door 70 (which is shown in open position in FIG. 1), and reservoir holder 72. The syringe main body 18 generally includes a transparent or translucent plastic cylinder having its open end 74 connected to console 12. A closed end 76 of syringe main body 18 contains two ports: inlet port 78 and outlet port 79. Plunger/piston 20 is movable within syringe main body 18. Plunger/piston 20 is connected to, and driven by a motor located within console 12.

The contrast media reservoir 22 is connected through inlet valve system 24 to inlet port 78. Radiographic contrast material is drawn from reservoir 22 through inlet valve system 24 and inlet port 78 into the pumping chamber defined by syringe main body 18 and plunger/piston 20. Inlet valve system 24 is a one-way valve which permits air to flow from syringe main body 18 back into reservoir 22, but will not permit radiographic contrast material to flow from syringe main body 18 to reservoir 22 when fully closed.

In FIG. 1, the outlet port 79 of syringe main body 18 is connected to manifold 26 by a conduit 80. Manifold 26 includes a spring biased spool valve which normally connects first fluid inlet (“transducer/saline port”) 82 and fluid outlet (“patient port”) 84. When contrast media is to be injected, the pressure of the contrast media causes the spool valve to change states so that outlet port 79 is connected to patient port 84 via second fluid inlet (“contrast media inlet port”) 81 and conduit 80. Other types of valves that selectively communicate between the contrast media and the saline can be used, including the elastomeric type valves described in Applicant's U.S. Pat. No. 7,617,837.

In the embodiment shown, high pressure tube 28 is a flexible tube which connects patient port 84 to catheter 30. A three-way stop-cock 34 is located at the distal end of tube 28. A rotatable Luer lock connector 86 is connected to stop-cock 34 and mates with Luer connector 88 at the proximal end of catheter 30. A stopcock 34 either blocks flow between tube 28 and catheter 30, permits flow, or connects medication port 32 to catheter 30 (for use when medication is to be delivered through catheter 30 to the patient).

When catheter 30 is in place in the patient, and an injection of contrast media is not taking place, pressure transducer 38 can monitor the blood pressure through the column of fluid which extends from catheter 30, tube 28, patient port 84, manifold 26, transducer/saline port 82, tubing 90, T-connector 36, and tubing 92. In the embodiment shown, transducer 38 has an associated stop-cock 40 which allows transducer 38 to be exposed to atmospheric pressure during calibration and also allows for removal/expulsion of trapped air so the dome chamber of transducer 38 can be flushed with saline.

Peristaltic pump 44 supplies saline solution from bag 50 through saline check valve 46, tubing 42, T-connector 36 and tubing 90 to saline port 82. When peristaltic pump 44 is operating to supply saline solution, the saline solution is supplied through manifold 26 to patient port 84 and then through tube 28 to catheter 30. Peristaltic pump 44 also operates in an opposite direction to draw fluid from catheter 30 and through tube 28, manifold 26, tubing 90, T-connector 36 and tubing 42 to waste check valve 48 and then into waste collection bag 52. As mentioned above, saline may be alternatively delivered to the patient with a syringe system instead of a peristaltic pump.

In use, the user (typically a physician) enters into system 10 the safety parameters that will apply to the injection of radiographic contrast material. These safety parameters typically include the maximum amount of radiographic contrast material to be injected during any one injection, the maximum flow rate of the injection, the maximum pressure developed within syringe main body 18, and the maximum rise time or acceleration of the injection. To actuate an injection of contrast material, the user operates remote control 14 by squeezing trigger 66. Within the preset safety parameters, system 10 causes the flow rate of the injection to increase as the force or distance of travel of trigger 66 is increased.

For purposes of illustration, representative operations of system 10 will now be described, including contrast fill, air purge, and patient inject operations. Of course, system 10 can also be configured to perform many other types of operations including, for example, saline flush and patient pressure monitoring operations.

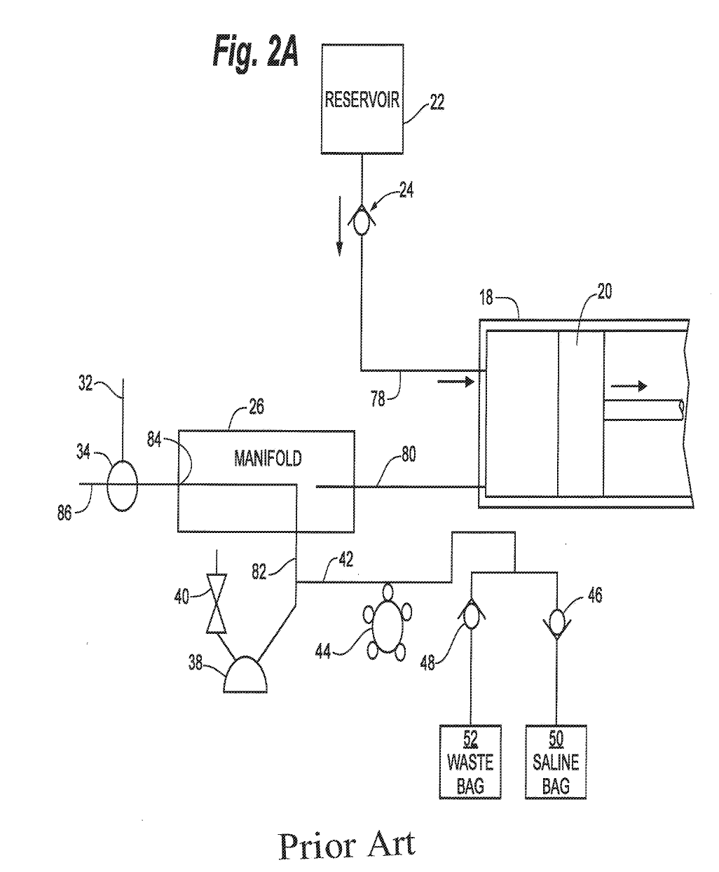

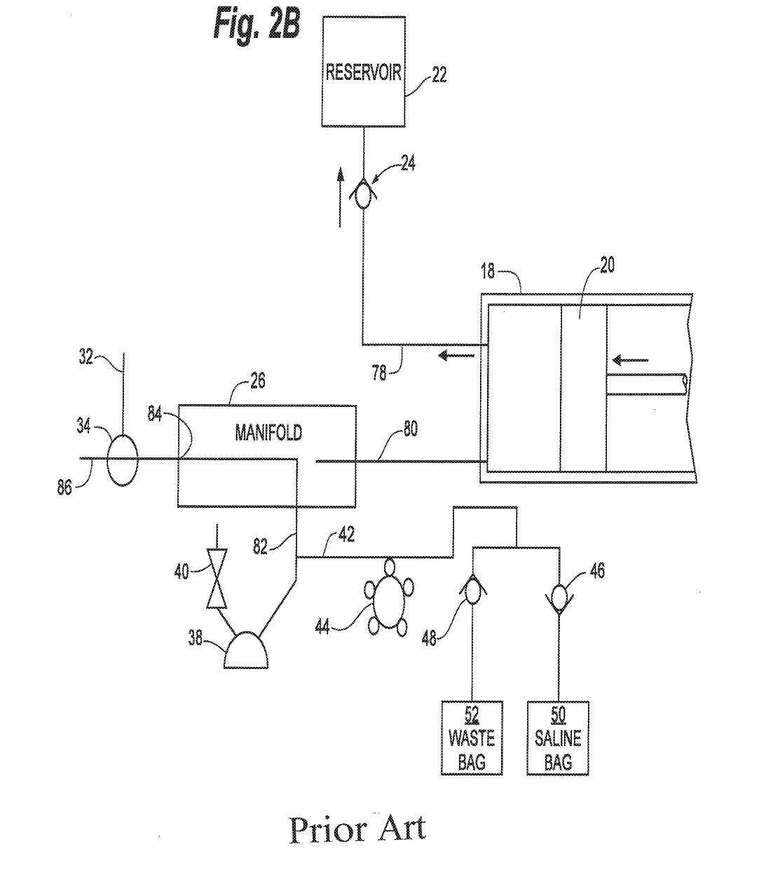

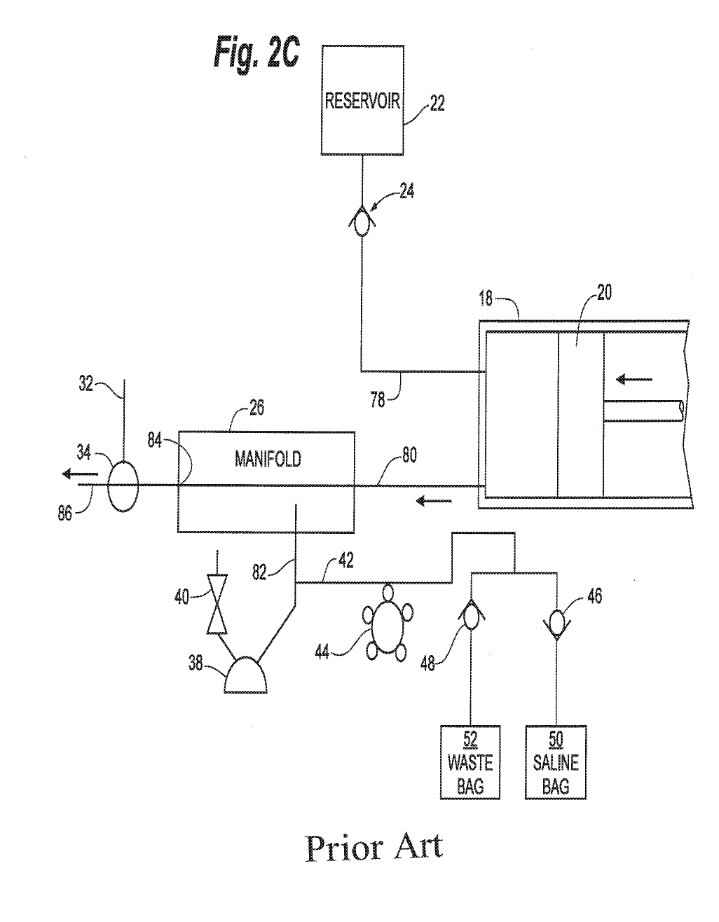

The contrast fill operation illustrated in FIG. 2A involves the filling of syringe main body 18 with contrast media from reservoir (contrast media supply) 22. The contrast fill operation is performed during initial set up of system 10, and may be repeated during operation of system 10 whenever syringe main body 18 is running low on radiographic contrast material. During initial set up of the system, plunger/piston 20 is initially driven to its furthest forward position adjacent closed end of syringe main body 18. This will expel to the atmosphere the majority of the air which is located within syringe main body 18.

Plunger/piston 20 is then retracted, which creates a vacuum within syringe main body 18 which draws contrast material from reservoir 22 through inlet valve system 24 in syringe main body 18 through inlet port 78.

The contrast fill operation typically will result in some air being drawn into or remaining within syringe main body 18. It is important, of course, to prevent air from being injected into the patient through catheter 30. The location of two ports at different elevations allows for a greater amount of safety in preventing air bubbles in the injection. Further, in some embodiments, the syringe can be placed at an angle relative to horizontal (e.g., about 10 degrees from horizontal), such that its closed end, and inlet port 78, are at a higher elevation than its open end. Such an embodiment facilitates air removal from the syringe through inlet port 78.

During the air purge operation, as illustrated in FIG. 2B, plunger/piston 20 travels forward to expel trapped air within syringe main body 18. The air, being lighter than the contrast media, gathers near the top of syringe main body 18. As plunger/piston 20 moves forward, the air is expelled from syringe main body 18 through inlet port 78 and inlet valve system 24. In the embodiment illustrated in FIG. 2B, inlet valve system 24 allows flow of contrast media from reservoir 22 to inlet port 78, but will not allow contrast media to flow in the opposite direction from inlet port 78 to reservoir 22. Inlet valve system 24 will, however, allow air to flow from port 78 to reservoir 22 until sufficient pressure builds in the syringe to close the inlet valve system.

FIG. 2C illustrates a patient inject operation. In this operation, plunger/piston 20 travels forward under the interactive control of the user, who is controlling trigger 66 of remote control 14. The movement of plunger/piston 20 creates hydraulic pressure to force contrast material out of syringe main body 18 through outlet port 79 and through manifold 26 and high pressure tube 28 into catheter 30. As shown in FIG. 2C, syringe outlet port 79 and patient port 84 are connected via contrast media inlet port 81 and conduit 80 for fluid flow during the patient inject operation.

In the embodiments shown, manifold 26 contains a valve which controls the routing of fluid connections between patient port 84 and either syringe outlet port 79 or transducer/saline port 82. As shown, manifold 26 can include a spool valve which is spring biased so that patient port 84 is normally connected to transducer/saline port 82 (as illustrated in FIGS. 2A and 2B). When the pressure at syringe outlet port 79 builds with the movement of plunger/piston 20 forward, the bias force against the spool valve is overcome so that syringe outlet port 79 is connected to patient port 84, and transducer/saline port 82 is disconnected the valve within manifold 26 protects pressure transducer 38 from being exposed to the high pressure generated by the patient inject operation. The spool valve opens automatically during the patient inject operation in response to increase pressure exerted on it from the syringe outlet port 79. The spool valve closes and returns to its original position allowing for connection of patient port 84 to transducer 38 when a slight vacuum is applied by retraction of plunger/piston 20 at the end of each patient inject operation. In an alternative embodiment, the valve within manifold 26 is an electromechanical or motor driven valve which is actuated at appropriate times to connect either syringe outlet port 79 or transducer/saline port 82 to patient port 84. In such embodiments, the actuator mechanism can be controlled by console 12. Once again in this alternative embodiment, the valve protects pressure transducer 38 from being exposed to high pressure.

The operation of the contrast injector system can be controlled by any suitable method. In general, the controls will include a digital computer which receives input signals from remote control 14 and front panel controls 56, and provides signals to display 58 to display operation data, alerts, status information and operator prompts, and controls the motion of plunger/piston 20 through a motor drive circuit with a motor.

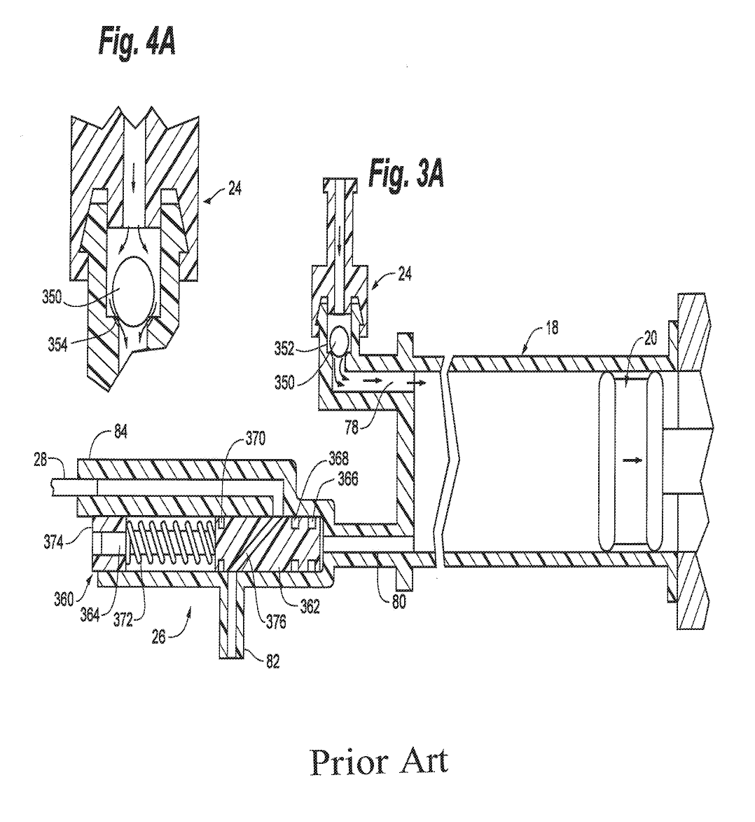

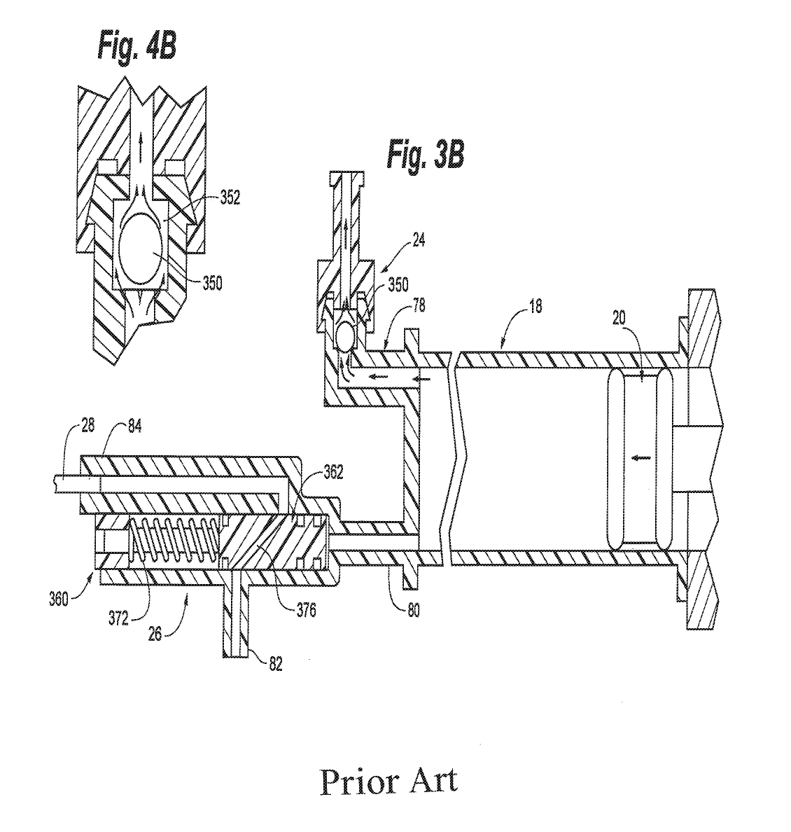

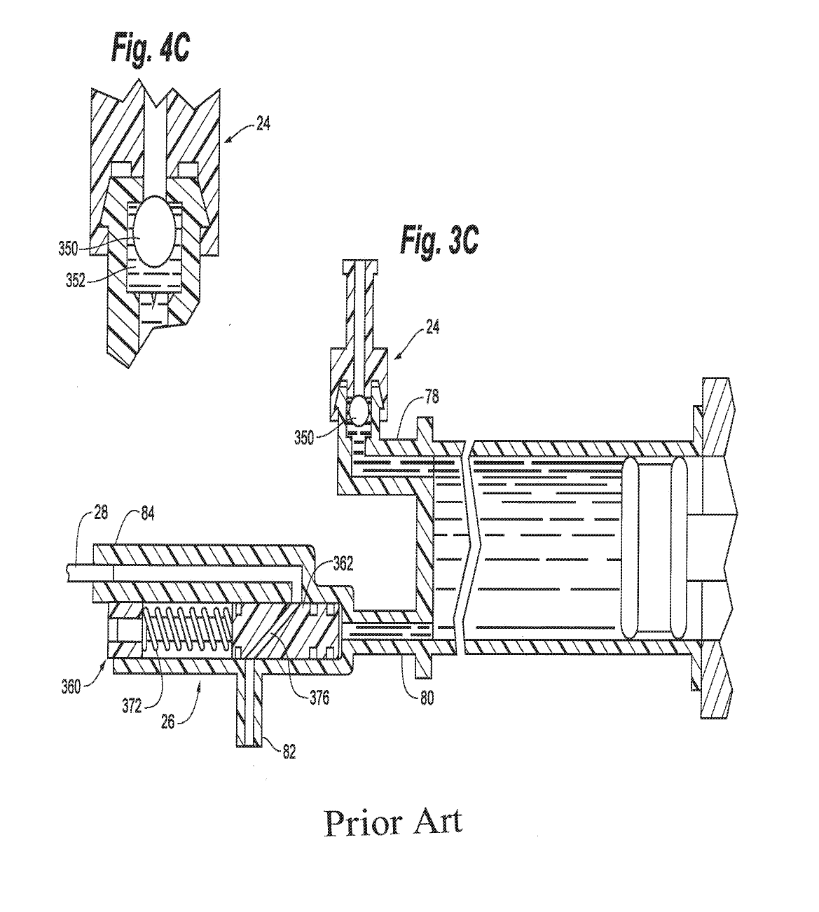

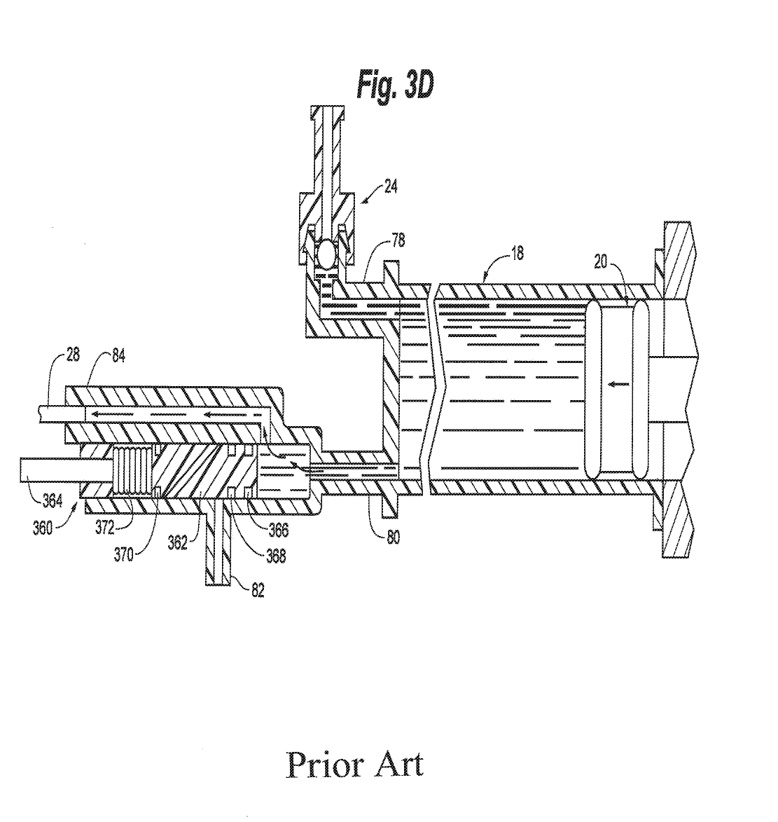

FIGS. 3A-3D and 4A-4C illustrate the general operation of an embodiment of an inlet valve system 24 and manifold 26 during contrast fill, air purge and patient injection operations.

FIGS. 3A and 4A illustrate an embodiment of an inlet valve system 24, manifold 26, syringe main body 18, and plunger/piston 20 during a contrast fill operation. As shown, inlet valve system 24 includes a valve member 350 which is positioned at a lower seated position within valve chamber 352 in FIGS. 3A and 4B. For purposes of illustration, valve member is represented as a ball in FIGS. 3A-4C. However, valve member 350 may include a wide variety of shapes and features. As shown, contrast media is being drawn into syringe main body 18 by the rearward movement of plunger/piston 20. The contrast material flows through passages 354 around valve member 350 and into inlet port 78.

As shown, manifold 26 contains main passageway 330, which includes a valve (“spring loaded spool valve”) 360. Furthermore, spring loaded spool valve 360 includes spool body 362, shaft 364, O-rings 366, 368 and 370, bias spring 372, and retainer 374. As shown in FIG. 3A, during the contrast fill operation, bias spring 372 urges spool body 362 to its right-most position toward syringe main body 18. In this position, spool body 362 blocks outlet port 79 of syringe main body 18 while connecting transducer saline port 82 to patient port 84 through diagonal passage 376. O-rings 366 and 368 on the one hand, and O-ring 370 on the other hand, are positioned on the opposite sides of diagonal passage 376 to provide a fluid seal.

FIGS. 3B and 4B illustrate an embodiment of an air purge operation. Syringe main body 18 has been filled with contrast fluid, but also contains trapped air. Plunger/piston 20 is driven forward to force the air out of syringe main body 18 through inlet port 78 and through inlet valve system 24 around the valve member. During the air purge operation, spool valve 360 is in the same position as in FIG. 3A. Diagonal passage 376 connects transducer saline port 82 with patient port 84. As a result pressure monitoring by pressure transducer 38 can be performed during the air purge (as well as the contrast fill) operation.

FIGS. 3C and 4C illustrate the state of manifold 26 and inlet valve system 24 at the end of the air purge operation and at the beginning of a patient inject operation. In FIG. 3C, all air has been expelled from syringe main body 18. Valve member 350 may float on the radiographic contrast material, so that when all air has been removed and the radiographic contrast material begins to flow out of syringe main body 18 and through inlet port 78 to valve chamber 352, valve member 350 is moved upwards to its upper seated position. Valve member 350 blocks any continued upward flow of contrast media, as is illustrated in FIGS. 3C and 4C.

In the state which is illustrated in FIG. 3C, the pressure within syringe main body 18, and specifically the pressure in outlet port 79 has not yet reached a level at which the bias force of spring 372 has been overcome. As a result, spool body 362 has not yet moved to the left and diagonal passage 376 continues to connect transducer saline port 82 with patient port 84.

FIG. 3D illustrates an embodiment of a patient inject operation. Plunger/piston 20 is moving forward, and inlet valve system 24 is closed. The pressure at outlet port 79 has become sufficiently high to overcome the bias force of spring 372. Spool body 362 has been driven to the left so that outlet port 79 is connected to patient port 84 through main passageway 330. At the same time spool body 362 blocks transducer/saline port 82. By virtue of the operation of spool valve 360, the high pressure generated by movement of plunger/piston 20 and syringe main body 18 is directly connected to patient port 84, while saline port 82 and pressure transducer 38 are protected from the high pressure. The pressure to actuate may be variable and determined after manufacture by increasing or decreasing the syringe preload.

FIG. 5A illustrates a perspective view of an embodiment of manifold 26. Manifold 26 includes contrast media inlet port 81, saline port 82, patient port 84, and main passageway 330. In some embodiments, main passageway 330 has selective fluid communication with contrast media inlet port 81, saline port 82, and patient port 84. Saline port 82 and patient port 84 may include one low pressure port and one high pressure port, respectively. In one example, pressures that may be experienced by high pressure port 84 include pressures of around 1200 psi. One or more (e.g., each) of the ports 81, 82, 84 can be integrally formed on the manifold 26, such as bonded directly to the manifold 26 main body. Contrast media inlet port 81 can be in fluid communication with outlet port 79 through conduit 80, such as shown in FIG. 5B.

FIG. 5B illustrates a cross-sectional view of an embodiment of a connection between conduit 80 and contrast media inlet port 81, while FIG. 5C illustrates a close-up perspective view of a portion of manifold 26 where the contrast media inlet port 81 is located (without the connection to conduit 81). The connection between the conduit 80 and port 81 can allow contrast media to be delivered from the syringe main body (e.g., via outlet port 79 of conduit 80) through manifold 26 (e.g., via main passageway 330 and patient port 84) and ultimately to the patient. As shown in the embodiment of FIG. 5B, contrast media inlet port 81 may include a female Luer fitting 490 in connection with a male Luer fitting 495 of conduit 80. Such connection may help to make a leak-free connection between the conduit 80 and port 81. In some examples, the female Luer fitting 490 on the port 81 may mate to a rotating male Luer fitting on the syringe main body (e.g., male Luer fitting 495 where conduit 80 is an integral part of the syringe main body).

The contrast media inlet port 81 can include threads 500 on an exterior surface 501, while an interior surface 502 of the port 81 can interface with the conduit 80. In particular, where a Luer connection is utilized, the interior surface 502 may in some instances define the female Luer fitting 490. Thus, the interior surface 502 of the port 81 can interface with the male Luer fitting 495 of conduit 80. In some embodiments, the threads 500 of the port 81 can engage with a locking nut 503 on the conduit 80. As the male Luer fitting 495 of the conduit 80 is inserted into the female Luer fitting 490 of contrast media inlet port 81, the locking nut 503 may engage with the threads 500 to further secure the connection between contrast media inlet port 81 and conduit 80. As shown in the example of FIG. 5B, the interior surface 502 of the port 81 can also include a surface finish 510 which will now be described further.

FIG. 5D illustrates a perspective view of an embodiment of contrast media inlet port 81 with the surface finish (“roughened inner surface finish”) 510. In some embodiments, contrast media inlet port 81 and conduit (e.g., portion of the conduit defining the outlet port) may have smooth surfaces. For instance, in some embodiment both the conduit and locking nut may be made from a polycarbonate material, and thus may include one or more highly polished polycarbonate surfaces. These embodiments can result in connections that are not completely secure when high pressure fluid is communicated. To withstand this high pressure and prevent soft connections, leaks, and disconnections, the surface finish 510 may be applied to contrast media inlet port 81 to allow for higher friction and retention forces between the smooth surface of the conduit (e.g., surface of the conduit defining the male Luer fitting and the outlet port) and the surface finish 510 on the interior surface of port 81.

The surface of the port 81 (e.g., interior surface of port 81) that is to interface with the male Luer fitting, or other surface, of the conduit can be treated so as to include the surface finish 510. Thus, in this example the female Luer fitting would include the surface finish 510. When such surface of the port is treated, peaks and valleys on the surface of the port 81 can be created (such as seen in the illustration of the exemplary surface finish 510 in FIG. 5B). For instance, in some embodiments the surface finish 510 can include a textured surface finish of a series of peaks and valleys that generally alternate around a circumference of the surface (e.g., interior surface of port 81). As one example, the valleys in the alternating series of peaks and valleys can be at an elevation on the surface of the component (e.g., interior surface of the port) similar to an elevation of the surface prior to including the surface finish 510, while the peaks can be at an elevation protruding out above the elevation of the surface prior to including the surface finish 510. In such an example, such as shown in FIG. 5B, the peaks of the surface finish 510 will contact an outer surface of the interfacing component (e.g., male Luer fitting 495 of conduit 80). In another example, the valleys can be at an elevation on the surface of the component (e.g., interior surface of the port) below an elevation of the surface prior to including the surface finish 510, while the peaks can be at an elevation similar to an elevation of the surface prior to including the surface finish 510.

In one embodiment, the surface finish 510 having the peaks and valleys can define a surface roughness of greater than 50 micro inches (1.27 micro meters). In another embodiment, the surface finish 510 can define a surface roughness of between 50 and 125 micro inches (between 1.27 and 3.18 micro meters). In a further embodiment, the surface finish 510 can define a surface roughness of between 60 and 90 micro inches (between 1.52 and 2.29 micro meters). In yet a further embodiment, the surface finish 510 can define a surface roughness of between 75 and 85 micro inches (between 1.91 and 2.16 micro meters). In such embodiments, the surface roughness defined by the surface finish 510 can be substantially uniform around a perimeter of the surface having the surface finish 510 (e.g., around a circumference of the interior surface of port 81). In these embodiments, the surface finish 510 can further be included along a length of the surface on which it is included (e.g., around the circumference of the interior surface of port 81 along a length of the interior surface of port 81 from an end of port 81 to an opposite end of port 81 at which port 81 communicates with the main passageway of defined by the manifold).

Including the surface finish 510 on the surface of the port 81 with the peaks and valleys thereon can provide benefits during pressurized fluid delivery through the manifold. For instance, the peaks of the rougher surface (e.g., interior surface of port 81 having surface finish 510) may penetrate to the base material of the conduit (e.g., at the male Luer fitting defining the outlet port) and deform under pressure allowing for a more complete seal. Further, in some additional embodiments an interior surface of the male Luer fitting can also include a similar surface finish as that of the surface of the port 81.

The surface may be provided by any suitable method. In some embodiments, it is cast in a mold, such that, for example, the interior surface of port 81 is textured to include the described surface finish 510 via a molded in texture. In other embodiments, the surface finish 510 is applied to a smooth surface of the manifold, also via a molded in texture. In certain embodiments, the surface treatment is applied with a tool, such as a rotary tool. In one example, the treated surface can be created using a rotary tool fitted with a fine finish diamond bit. In this example, the fine finish diamond bit can create a textured surface of peaks and valleys to increase friction and retention forces with an opposing surface.

Surface finish 510 may provide for better connections. Surface finish 510 may create a fluid path to aid in displacement of fluids. Further, treated surface finish 510 can create a dissimilar surface finish that may initially reduce contact area between a manifold port and a conduit, such as a male portion of a Luer connection. This reduced contact area can prevent premature sticking or bonding between two smooth, semi-rigid materials.

In some embodiments, manifold 26 may be connected with syringe main body 18. In one embodiment, this connection can be made by inserting conduit 80 into contrast media inlet port 81. Conduit 80 may be a male Luer fitting that can connect with contrast media inlet port 81, which may be a female Luer fitting. Alternatively, contrast media inlet port 81 may be a male Luer fitting that can connect with conduit 80, which may be a female Luer fitting. In other embodiments, the connection between conduit 80 and contrast media inlet port 81 can be secured by screwing locking nut 503 onto screw threads 500. Locking nut 503 may be attached to conduit 80 so that when it screws onto screw threads 500, it connects conduit 80 to contrast media inlet port 81.

The foregoing description addresses examples encompassing the principles of various embodiments of the present invention. The embodiments may be changed, modified and/or implemented using various types of arrangements. In particular, one or more embodiments may be combined in a single inlet valve system. Those skilled in the art will readily recognize various modifications and changes that may be made to these embodiments of the invention without strictly following the exemplary embodiments and applications illustrated and described herein, and without departing from the scope of the invention. Accordingly, it is not intended that the invention be limited, except as by the appended claims.

1. A fluid connection assembly comprising:
a conduit for communicating fluid; and
a manifold having a main body defining a main passageway, wherein the conduit is connected to the manifold, and wherein the manifold comprises:
a first fluid inlet and a second fluid inlet in fluid communication with the main passageway;
a fluid outlet in fluid communication with the main passageway;
a valve, wherein the valve is configured to switch between allowing fluid communication through either the first fluid inlet or the second fluid inlet, and the fluid outlet; and
wherein at least one of the first fluid inlet, the second fluid inlet, and the fluid outlet comprises a textured surface finish that includes a series of alternating peaks and valleys around a circumference of the at least one of the first fluid inlet, the second fluid inlet, and the fluid outlet, and wherein the textured surface finish is in contact with the conduit.

2. The fluid connection assembly of claim 1, wherein the first fluid inlet is connected to a supply of saline.

3. The fluid connection assembly of claim 1, wherein the second fluid inlet is connected to a supply of contrast media.

4. The fluid connection assembly of claim 1, wherein the fluid outlet delivers fluid to a patient.

5. (canceled)

6. (canceled)

7. The fluid connection assembly of claim 1, wherein the textured surface finish defines a surface roughness of between 50 and 125 micro inches.

8. The fluid connection assembly of claim 1, wherein the second fluid inlet comprises the textured surface finish.

9. The fluid connection assembly of claim 1, wherein the first fluid inlet, the second fluid inlet, and the fluid outlet each comprises an inner surface, wherein at least one of the inner surfaces comprises the textured surface finish.

10. The fluid connection assembly of claim 1, wherein at least one of the first fluid inlet, the second fluid inlet, or the fluid outlet comprises screw threads on a surface opposite the textured surface finish.

11. The fluid connection assembly of claim 10, wherein the screw threads engage with a locking nut.

12. The fluid connection assembly of claim 1, wherein the manifold comprises a polycarbonate body.

13. The fluid connection assembly of claim 1, wherein the second fluid inlet is configured to withstand pressures of around 1200 psi.

14. The fluid connection assembly of claim 1, wherein the valve comprises a spool valve, wherein the spool valve is spring biased so that the first fluid inlet is normally connected to the fluid outlet, wherein bias force against the spool valve is overcome so that the second fluid inlet is connected to the fluid outlet.

15. The fluid connection assembly of claim 1, wherein the first fluid inlet comprises a pressure transducer.

16. The fluid connection assembly of claim 1, wherein the conduit includes a male Luer connection fitting.

17. A fluid connection assembly comprising:
a conduit for communicating fluid; and
a manifold having a main body defining a main passageway, wherein the conduit is connected to the manifold, and wherein the manifold comprises:
a first fluid inlet and a second fluid inlet in fluid communication with the main passageway;
a fluid outlet in fluid communication with the main passageway;
a valve, wherein the valve is configured to switch between allowing fluid communication through either the first fluid inlet or the second fluid inlet, and the fluid outlet;
wherein the second fluid inlet comprises an inner surface, wherein the inner surface comprises a surface finish defining a surface roughness of more than about 50 micro inches, and wherein the surface finish is in contact with the conduit; and
wherein the second fluid inlet comprises an outer surface, wherein the outer surface comprises screw threads.

18. A method of connecting a fluid conduit to a manifold comprising:
placing a surface of a fluid conduit in contact with a surface of a manifold having a surface finish that includes a series of alternating peaks and valleys around a circumference and along a length of the surface of the manifold, the manifold having a main body defining a main passageway, a first fluid inlet and a second fluid inlet in fluid communication with the main passageway, a fluid outlet in fluid communication with the main passageway, and a valve configured to switch between allowing fluid communication through either the first fluid inlet or the second fluid inlet, and the fluid outlet, wherein at least one of the first fluid inlet and the second fluid inlet includes the surface finish; and
connecting the fluid conduit with the at least one of the first fluid inlet and the second fluid inlet including the surface finish, wherein connecting the fluid conduit comprises securing the fluid conduit to multiple threads spaced apart along a length of a surface of the manifold that is opposite the surface of the manifold having the surface finish, and wherein the fluid conduit is secured to the multiple threads such that the surface of the fluid conduit in contact with the surface finish extends within the at least one of the first fluid inlet and the second fluid inlet beyond the multiple threads.

19. (canceled)

20. The method of claim 18, wherein the fluid conduit is connected to the second fluid inlet, and further comprising injecting a fluid from the second fluid inlet through the fluid outlet at a pressure of about 1,200 psi.

21. The fluid connection assembly of claim 1, wherein the series of alternating peaks and valleys around the circumference of the at least one of the first fluid inlet, the second fluid inlet, or the fluid outlet further extends along a length of the at least one of the first fluid inlet, the second fluid inlet, or the fluid outlet.

22. The fluid connection assembly of claim 9, wherein the conduit comprises a smooth outer surface that is in contact with the at least one of the inner surfaces having the textured surface finish.

23. The fluid connection assembly of claim 1, wherein the textured surface finish is on an inner surface of the second fluid inlet, and wherein the peaks are at an elevation protruding out from an elevation of the inner surface at a location free from the textured surface finish.
